(19) United States
(12) Patent Application Publication
(10) Pub. No.: US 2019/0131812 A1
(43) Pub. Date: May 2, 2019

(54) PORTABLE ELECTRONIC DEVICE

(71) Applicant: LG ELECTRONICS INC., Seoul (KR)

(72) Inventors: Yongho LEE, Seoul (KR); Junho YANG, Seoul (KR)

(73) Assignee: LG ELECTRONICS INC., Seoul (KR)

(21) Appl. No.: 15/748,832

(22) PCT Filed: Nov. 17, 2015
(86) PCT No.: PCT/KR2015/012310

§ 371 Date: Jan. 30, 2018

(30) Foreign Application Priority Data
Sep. 3, 2015 (KR) 10-2015-0124764

(51) Int. Cl. H02J 7/02; H05B 33/08; H05B 37/02; H05K 5/00; G06F 1/16; A61B 5/0404; A61B 5/00

(52) U.S. Cl. CPC H02J 7/025; H05B 33/0857; H05B 37/0272; H05K 5/0004; A61B 5/6826; G06F 1/163; A61B 5/0404; A61B 5/4872; H05K 5/0086

(57) ABSTRACT

A portable electronic device, which allows for easy self-charging while minimizing the size of a battery and can thus achieve long-term use, comprises: a ring-shaped first housing; a second housing which has a ring shape corresponding to the first housing and is arranged to be parallel to a side of the first housing; a charging coil mounted on the first housing and wound along the ring shape of the first housing; a plurality of magnets arranged in the second housing; and a battery which is charged using a current flowing through the charging coil when the second housing is rotated with respect to the first housing.

### TECHNICAL FIELD

The present invention relates to a portable electronic device using rotatory power of a ring.

### BACKGROUND ART

Electronic devices may be generally classified as stationary electronic devices or portable electronic devices according to their mobility. Various types of portable electronic devices are currently used including a portable terminal capable of making a call using mobile communication such as a cellular phone and a smartphone, a portable audio device such as a portable CD player and MP3, a notebook, a tablet PC, and the like.

Recently, in order to make a user more easily carry a portable electronic device, a size of the portable electronic device is more reduced. In particular, a portable electronic device capable of being worn on a body of a user is appearing. For example, wearable electronic devices such as a bracelet/watch-type communication device, a Bluetooth earphone, and a sunglass-type electronic device capable of being worn on a body of a user are appearing.

However, since it is difficult to provide power to a portable electronic device using a wire, the portable electronic device operates using stored power in a manner of being equipped with its own battery. In this case, since a size of the battery relates to hours of use, if a high-capacity battery is used, it may have a problem that a size of the portable electronic device increases. Hence, to secure hours of use of the electronic device is turned out as an important task.

### DISCLOSURE OF THE INVENTION

### Technical Problem

An object of the present invention is to provide an electronic device capable of minimizing capacity of a battery of the portable electronic device and performing self-charging to secure hours of use.

### Technical Solution

To achieve these and other advantages and in accordance with the purpose of the present invention, as embodied and broadly described, according to one embodiment, a portable electronic device includes a first housing of a ring shape, a second housing having a ring shape corresponding to the first housing and is arranged to a side of the first housing in parallel, a charging coil embedded in the first housing and wound along the ring shape of the first housing, a plurality of magnets deployed to the second housing, and if the second housing rotates with respect to the first housing, a battery charged using a current flowing through the charging coil.

The portable electronic device can further include ball bearings which are positioned between the first housing and the second housing to assist the second housing to rotate.

An inner side of the second housing includes a material of low friction force and an inner side of the first housing can include a material of high friction force.

A size of a circle formed by an inner side of the second housing can be configured to be greater than a size of a circle formed by an inner side of the first housing.

An inner side of the second housing may have a slope inclined to an outer side of the second housing as getting far from the first housing.

The second housing can be deployed to both sides of the first housing as a pair.

The portable electronic device includes a first part mounted in a manner of being adjacent to an outer side of the first housing or the first part mounted in a manner of being exposed to the outer side, a second part mounted in a manner of being adjacent to an inner side of the first housing or the second part mounted in a manner of being exposed to the inner side, and a flexible board configured to connect the first part, the second part, and the battery while being positioned at the inside of the first housing.

In this case, a part of the flexible board overlapped with the first part is bent in a direction of the inner side of the first housing and a part of the flexible board overlapped with the second part can be bent in a direction of the outer side of the first housing.

The flexible board includes an insulation film, a first electrode formed on one side of the insulation film, and a second electrode formed on one side of the insulation film in a manner of being separated in a length direction of the insulation film. In this case, the first electrode and the second electrode are respectively contacted with the first part and the second part in a manner that the insulation film is twisted between the first electrode and the second electrode to make the first electrode and the second electrode face an opposite direction.

The portable electronic device can further include a plurality of body fat measuring terminals formed on an outer side of the first housing.

The portable electronic device can further include an electrocardiogram sensor formed on an inner side of the first housing.

The electrocardiogram sensor can include a pair of electrocardiogram sensor terminals deployed to the inner side of the first housing to make the terminals face each other.

The portable electronic device can further include a wireless communication unit configured to connect an external terminal, a plurality of LEDs mounted on an outer side of the first housing, and if event information is received via the wireless communication unit, a controller configured to control the event information to be provided to a user by controlling color, a turn-on order, a turn-on count, and the like of a plurality of the LEDs.

The portable electronic device can further include a controller configured to supply a current flowing through the charging coil to the battery in a charging mode and the controller configured to control the portable electronic device according to a direction or an amount of the current flowing through the charging coil in a user input mode.

The portable electronic device can further include a wireless communication unit configured to connect an external terminal. In this case, if event information is received via the wireless communication unit, the controller can switch a mode into the user input mode.

### Advantageous Effects

According to the present invention, since it is able to minimize a battery size of a portable electronic device and simply perform self-charging, it is able to use the portable electronic device for a long time.

According to the present invention, since a sensor located at the inside of a portable electronic device is combined with a body of a user in a manner of being adhere to the body, it is able to consistently measure bio-information such as an electrocardiogram, a body temperature, and the like.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DESCRIPTION OF DRAWINGS

FIG. 1 is a block diagram for explaining a portable electronic device according to the present invention;

FIG. 2 is a perspective diagram for a portable electronic device according to the present invention;

FIG. 3 is a cross-sectional diagram for a portable electronic device in length direction according to the present invention;

FIG. 4 is an exploded diagram for a portable electronic device according to the present invention;

FIG. 5 is a cross-sectional diagram for a portable electronic device in width direction according to the present invention;

FIG. 6 is a diagram illustrating a usage example of a portable electronic device according to the present invention;

FIG. 7 is a diagram illustrating a flexible board of a portable electronic device according to the present invention.

### BEST MODE

### Mode for Invention

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, when an element is referred to as being “directly connected with” another element, there are no intervening elements present.

A singular representation may include a plural representation unless it represents a definitely different meaning from the context.

Terms such as “include” or “has” are used herein and should be understood that they are intended to indicate an existence of several components, functions or steps, disclosed in the specification, and it is also understood that greater or fewer components, functions, or steps may likewise be utilized.

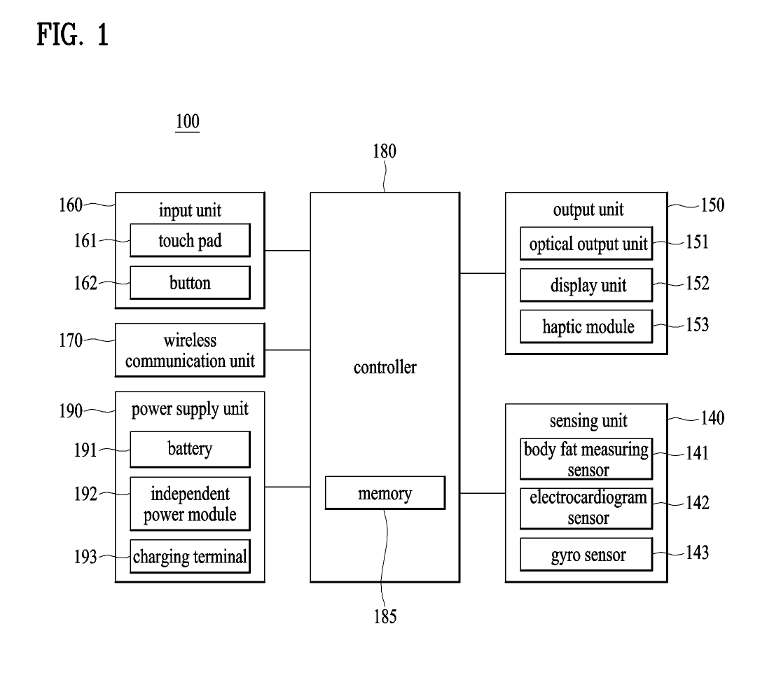

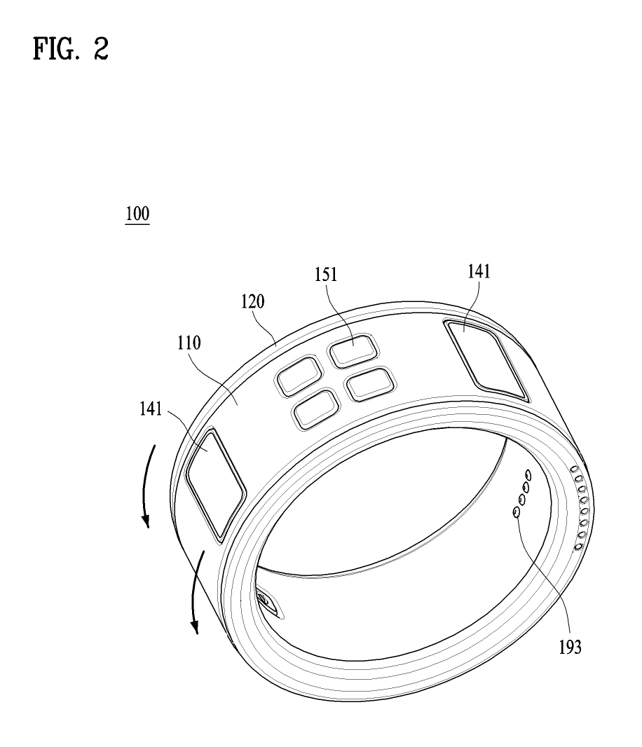

FIG. 1 is a block diagram for explaining a portable electronic device 100 according to the present invention and FIG. 2 is a conceptual diagram for a portable electronic device 100 according to the present invention seen from a different direction.

The portable electronic device 100 can include housings 110/120 constructing the exterior of the portable electronic device, a wireless communication unit 170 mounted on the housings 110/120, an input unit 160, a sensing unit 140, an output unit 150, a controller 180, and a power supply unit 190.

It is understood that implementing all of the illustrated components in The FIG. 1 is not a requirement, and that greater or fewer components may alternatively be implemented.

More specifically, the wireless communication unit 170 typically includes one or more modules which permit wireless communications between the portable electronic device 100 and a wireless communication system, communications between the portable electronic device 100 and another portable electronic device, communications between the portable electronic device 100 and an external server. Further, the wireless communication unit 170 typically includes one or more modules which connect the portable electronic device 100 to one or more networks.

To facilitate such communications, the wireless communication unit 170 includes at least one selected from the group consisting of a broadcast receiving module, a mobile communication module, a wireless Internet module, a short-range communication module, and a location information module.

The sensing unit 140 is typically implemented using one or more sensors configured to sense internal information of the portable electronic device 100, the surrounding environment of the portable electronic device 100, user information, and the like.

For example, the sensing unit 140 may include at least one selected from the group consisting of a touch sensor, an acceleration sensor, a magnetic sensor, a G-sensor, a gyroscope sensor 143, a motion sensor, an RGB sensor, a finger scan sensor, a ultrasonic sensor, an optical sensor, a microphone, a battery gauge, an environment sensor (e.g., a barometer, a hygrometer, a thermometer, a radiation detection sensor, a thermal sensor, a gas sensor, etc.), and a biometric sensor such as a body fat measuring sensor 141 and an electrocardiogram sensor.

Meanwhile, the portable electronic device 100 disclosed in the present specification may be configured to utilize information obtained from two or more sensors of the sensing unit 140, and combinations thereof.

The output unit 150 is typically configured to output various types of information, such as audio, video, tactile output, and the like. The output unit 150 can include at least one selected from the group consisting of an optical output unit 151, a display unit 152, an audio output unit 153, and a haptic module 153.

The optical output unit 151 consists of a plurality of lamps. In case of using LED, it may variously control color and brightness. The optical output unit 151 can provide not only a design function but also various informations according to color and brightness. For example, if a text message is received, the optical output unit is turned on in green color. If a phone call is received, the optical output unit can be turned on in blue color.

The display unit 152 displays (outputs) information processed in the portable electronic device 100. For example, the display unit 152 can display execution screen information of an application program executed in the portable electronic device 100 or UI (user interface) and GUI (graphic user interface) information according to the execution screen information.

The display unit 152 can include a touch sensor configured to sense a touch touched on the display unit 152 to receive a control command input by a touch scheme. If a touch is inputted on the display unit 152, the touch sensor senses the touch and the controller 180 can generate a control command in response to the touch. Contents inputted by a touch scheme may correspond to a text message, a number, or a menu item capable of being indicated or designated in various modes.

Meanwhile, the touch sensor can be deployed between a window 151a and a display (not depicted) mounted on a rear side of the window 151a in a manner of being configured in a film form equipped with a touch pattern. Or, the touch sensor may correspond to a metal wire directly patterned on the rear side of the window 151a. Or, the touch sensor and the display can be configured as a unibody. For example, the touch sensor can be mounted on a board of the display or can be embedded in the display. The display unit 152 may form a touch screen together with the touch sensor. In this case, the touch screen may function as a user input unit.

The input unit 160 can include a user input unit for inputting a control command of a user similar to a touch pad 161 and a button 162. Besides, the input unit can include a camera for inputting a video signal or a microphone for inputting an audio signal. An audio data or an image data collected by the input unit 160 is analyzed and can be processed as a control command of a user.

The controller 180 typically functions to control overall operation of the portable electronic device 100, in addition to the operations associated with the application program. The controller 180 may provide or process information or functions appropriate for a user by processing signals, data, information and the like, which are inputted or outputted by the aforementioned various components, or activating application programs stored in the memory 185.

The controller 180 controls some or all of the components mentioned earlier in FIG. 1 to drive an application program stored in the memory 185. Moreover, in order to drive the application program, the controller 180 may operate two or more components among the components included in the portable electronic device 100 by combining the components with each other.

The memory 185 is typically implemented to store data to support various functions of the portable electronic device 100. For instance, the memory 170 may be configured to store a plurality of application programs (or, applications) executed in the portable electronic device 100, data or instructions for operations of the portable electronic device 100, and the like. Some of these application programs may be downloaded from an external server via wireless communication.

The power supply unit 190 receives external power or internal power under the control of the controller 180 and supplies power to each of the components included in the portable electronic device 100. The power supply unit 190 may include a battery 191, and the battery 191 may be configured to be embedded in the portable electronic device, or configured to be detachable from the portable electronic device. In order to charge the battery 191, a charging terminal 193 configured to be connected with an external power source can be installed in a manner of being exposed to an outer side of a housing. According to the present invention, in order to use the portable electronic device 100 even in a situation that charging is unavailable, the portable electronic device 100 can further include an independent electric power module.

At least a part of the components may cooperate to operate the portable electronic device 100, control the portable electronic device 100, or implement a control method according to various embodiments described in the following. The operation, the control, or the control method of the portable electronic device 100 can be implemented on the portable electronic device 100 by driving at least one or more applications stored in the memory 185.

The portable electronic device 100 according to the present invention is explained in more detail with reference to FIG. 2 in the following. The portable electronic device 100 according to the present invention has a ring shape of which the center is empty and can be worn on a finger of a user. If a size of the ring is increased, the ring can also be worn on an arm. Yet, the present embodiment is explained on the basis of a ring-type portable electronic device 100 configured to be worn on a finger.

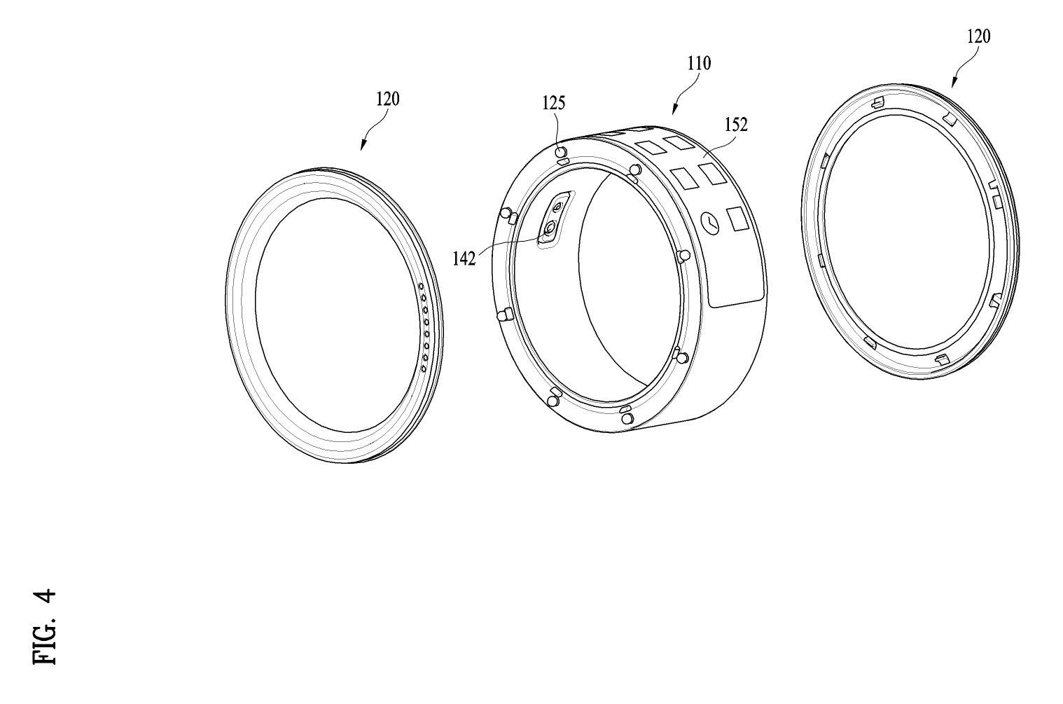

According to the present invention, an optical output unit 151 or a display unit 152 (refer to FIG. 4) configured to provide information to a user can be positioned at the outer side of the portable electronic device 100. The optical output unit 151 is turned on according to information received by the wireless communication module 170 and can provide various informations according to color and brightness. Referring to FIG. 2, although the optical output unit 151 is configured by 4 LED lamps, by which the present invention may be non-limited. The number of lamps and the arrangement of the lamps may vary.

The portable electronic device 100 may further include a display unit 152 in addition to the optical output unit 151. In order to implement a curved surface, it may use OLED (organic light emitting diode). If the OLED is used as a display, since power consumption is big, a problem of quickly consuming a battery 191 may exist. In order to solve the problem, the display 152 can be implemented using E-ink.

It may be able to implement a touch screen by laminating the display unit 152 with a touch pad 161. Due to the characteristic of the ring-shape wearable terminal, the touch screen can be easily contacted with a body of a user. Hence, it may be able to selectively activate or deactivate a touch input function.

Unlike a general display, E-ink is flexible as much as certain level and is implemented by deploying micro capsules equipped with a black particle and a white particle between two electrode layers. Once the particles are moved, although the particles are not continuously electrified, a screen can be maintained. In particular, it is able to maintain standby power close to 0.

And, a body fat measuring sensor 141 for measuring body fat can be further installed in the outer side of the portable electronic device 100. The body fat measuring sensor 141 can calculate body fat by contacting two or more electrodes to a body of a user and measuring impedance by passing an electric current through the body of the user. If more parts are contacted with the body of the user, a more precise measurement result can be obtained. Yet, it may still be able to measure a body fat using two electrodes.

For example, when the portable electronic device 100 is worn on an index finger of a left hand, if one electrode of the body fat measuring sensor 141 is contacted with a different finger (e.g., a thumb) and another electrode of the body fat measuring sensor 141 is contacted with a right hand, an electric current is passed through two arms and an amount of body fat can be measured.

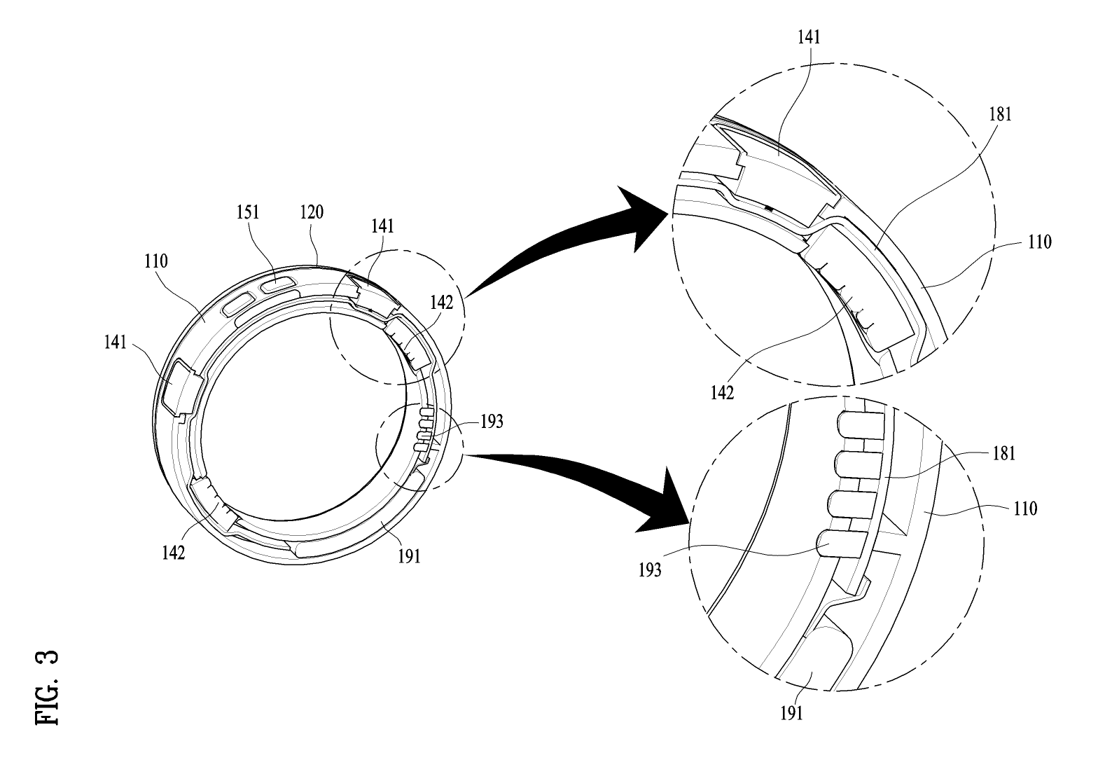

FIG. 3 illustrates a cross section cut along with the circumference of the portable electronic device shown in FIG. 2. A charging terminal 153 and an electrocardiogram sensor 142 can be installed in the inner side of housings 110/120.

The electrocardiogram sensor 142 can measure strength of a hear beat, a pulse, and the like by measuring an electric potential of a body surface which occurs when a heart beats. When an electrocardiogram is examined in general, in order to measure the electric potential, a pair of tongs is used to contact with a finger or a toe. Since the electrocardiogram sensor 142 according to the present invention is positioned at the inner side of the housing 110/120, if a portable electronic device is worn on a finger, the sensor can be adhere to the skin of a user. Since a pair of electrocardiogram sensors 142 is deployed in the inner side of the housing 110/120 in a manner of being divided to make the electrocardiogram sensors face each other, it may be able to obtain an effect similar to a case of picking up a finger using a tongs when a general electrocardiogram is measured.

Since it is able to perform user authentication using the electrocardiogram sensor 142, if the electrocardiogram sensor 142 is installed in the portable electronic device, it may be able to prevent the portable electronic device from being used by a third party. When a patient having a cardiac disorder wears the portable electronic device and consistently measures an electrocardiogram, if a result measured by the electrocardiogram sensor 142 is abnormal, the portable electronic device transmits a danger signal and may be able to give first aid to the patient.

A charging terminal 153 for charging an embedded battery 191 can be further installed in the inner side of the portable electronic device. If the charging terminal 153 is exposed to the outer side, since it is not aesthetically good, the charging terminal can be installed in the inner side of the portable electronic device. Yet, due to the characteristic of the portable electronic device 100, when capacity of the battery is insufficient, it is unable to immediately charge the battery. Hence, it may have an independent electric power system 1921/1922.

In general, an independent electric power device uses a method of converting kinetic energy into electric power using rotatory power. In general, when a spin structure is installed in a general electronic device, in most cases, a large space is required and a handle is protruded from an electronic device, thereby decreasing portability. On the contrary, since the portable electronic device 100 according to the present invention has housings 110/120 of a ring shape, it may be able to implement a spin structure using the housings.

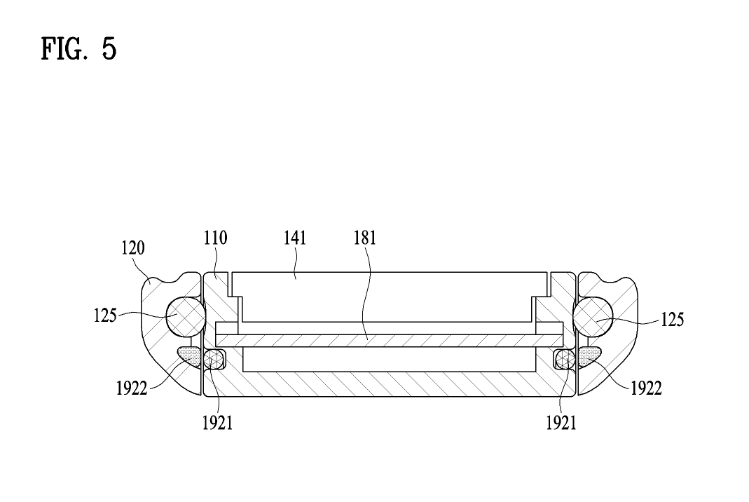

FIG. 4 is an exploded diagram for a portable electronic device 100 separated into a first housing 110 and a second housing according to the present invention and FIG. 5 is a cross-sectional diagram for a portable electronic device in width direction according to the present invention.

A housing of the present invention includes a first housing 110 equipped with electronic devices and a second housing 120 of a ring shape of a size corresponding to the second housing 120. The aforementioned optical output unit 151, the display unit 152, the battery 191, the controller 180, and the like can be deployed in the first housing. The second housing 120 is deployed in a side direction of the first housing 110 and is combined with the first housing 110 while the second housing is capable of rotating on the basis of the center of a ring.

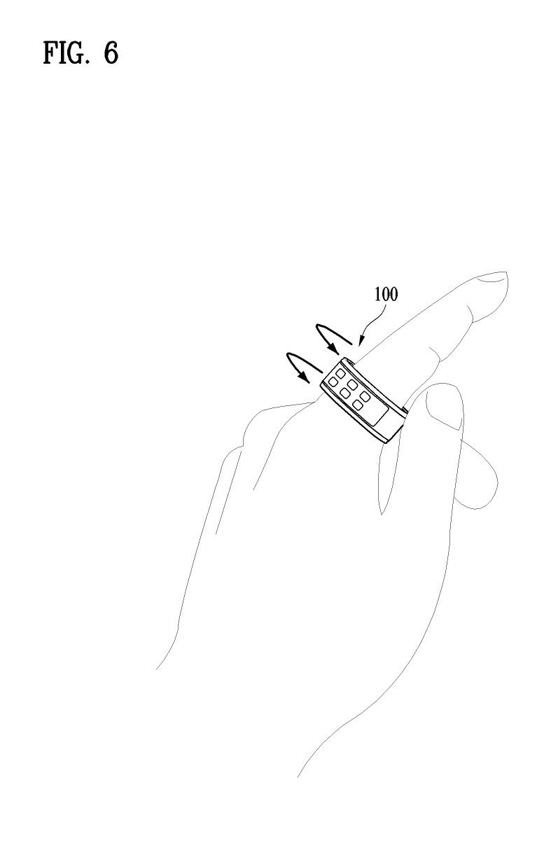

The second housing 120 is combined with the first housing 110 while the second housing is capable of relatively rotating. As shown in FIG. 4, a pair of second housings can be installed in the left side and the right side of the first housing. Or, the second housing can be installed in one side of the first housing only. When a pair of the second housings is installed in the left side and the right side of the first housing, a pair of the second housings may rotate at the same time in a manner of being connected with each other. Or, a pair of the second housings may separately rotate as well. FIG. 6 is a diagram illustrating a state that the portable electronic device 100 according to the present invention is worn on a user. The user wears the portable electronic device on a finger and may be able to rotate the second housing 120 using a different finger.

In order to make an inner side of the first housing 110 to be contacted with a finger of a user while the second housing is to be separated from the finger of the user, it may be able to configure a size of a circle formed by an inner side of the second housing to be greater than the first housing 110. Or, as shown in FIG. 5, it may be able to configure the inner side of the second housing to make a slope to make the inner side of the second housing 120 not to be contacted with the finger of the user.

In order to make the slope of the second housing 120 form a continuous side with the first housing 110, a part contacted with the first housing 110 may have a size identical to a size of the first housing 110. As getting far from the first housing 110, it may have a slope inclined to an outer side (top side in the drawing) of the second housing 120.

On the other hand, it may form a bump on the outer side of the second housing 120 to increase frictional force when a user pushes and rotates the second housing.

On the other hand, in order to maintain a state that the first housing 110 is fixed at a finger, it may use a material of high frictional force for the inner side of the first housing 110. Yet, if the friction force is too high, it is difficult to get the portable electronic device off from a finger. In particular, it may provide friction force as much as not rotating the first housing 110 when the second housing 120 rotates.

Or, it may use a material of low friction force for an opening direction of a ring and use a material of high friction force for a circumference direction of the ring. It may differently implement friction force according to a direction using a tiny bump direction on a surface.

In order to make the second housing 120 rotate for the first housing 110, ball bearings 125 can be deployed between the first housing 110 and the second housing 120. The ball bearings 125 correspond to materials of a ball shape interposed between two materials. In order to make the second housing 120 smoothly rotate for the first housing 110, the ball bearings 125 are rotating.

The first housing 110 includes a charging coil 1921 wound along with a ring of the first housing 110 and the second housing 120 includes magnets 1922 deployed along with a ring of the second housing 120. As shown in FIG. 5, the magnets 1922 can be deployed in the vicinity of the charging coil 1921. A plurality of the magnets 1922 can be deployed in a manner of being separated from each other with a prescribed distance.

If the second housing 120 rotates, positions of the magnets 1922 change and a magnetic field formed by the magnets 1922 changes. Induced current flows through the charging coil 1921 according to the change of the magnetic field and it may charge the battery 191 using the induced current flowing through the charging coil 1921.

The induced current can be flowed through the charging coil 1921 not only by a method of changing the magnetic field generated by the magnetics 1922 by rotating the second housing 120 but also by a method of settling down the portable electronic device on a wireless charging device. By doing so, it may be able to charge the battery 191.

According to the present invention, it may be able to input a user command using a current flowing through the charging coil 1921. The portable electronic device 100 can distinguish a charging mode from a user input mode. The portable electronic device supplies a current flowing through the charging coil 1921 to the battery 191 in the charging mode and can sense a rotation direction of the second housing 1210 using the current flowing through the charging coil 1921 in the user input mode.

A user can control the portable electronic device 100 according to a rotation direction of the second housing 120. For example, if the portable electronic device 100 is equipped with the display unit 152, the user can rotate the second housing 120 in a first direction or a second direction corresponding to an opposite direction of the first direction to move a screen of the display unit 152 or change a position of a selection window. If the portable electronic device is equipped with an audio output unit or is connected with an external audio output device via wireless communication, the user can adjust volume by rotating the second housing 120.

It may be able to input a different command according to a rotation direction, a rotation speed, and a rotation level of the second housing 120. If there are two second housings 120, it may be able to input a different command using each of the second housings 120.

A user can manually change the charging mode and the user input mode. If a specific event occurs and it is necessary to receive a user command input, a mode of the portable electronic device can be automatically changed to the user input mode.

The portable electronic device 100 according to the present invention can include an optical display unit, a display unit 152, a body fat measuring sensor, and the like positioned at the outer side of the portable electronic device 100 and includes a charging terminal, an electrocardiogram sensor 142, and the like positioned at the inner side of the portable electronic device 100. If the components are deployed in a manner of being overlapped, although it is able to deploy more components in the portable electronic device, thickness of the portable electronic device 100 increases, thereby degrading wearing sensation.

In order to decrease the thickness of the portable electronic device 100, as shown in FIG. 3, a component exposed to the outer side and a component exposed to the inner side can be deployed in a manner of being dislocated instead of being overlapped. A flexible board 181 connected with each of the components is deployed in the inside of the first housing 110 in a circumference direction of a ring.

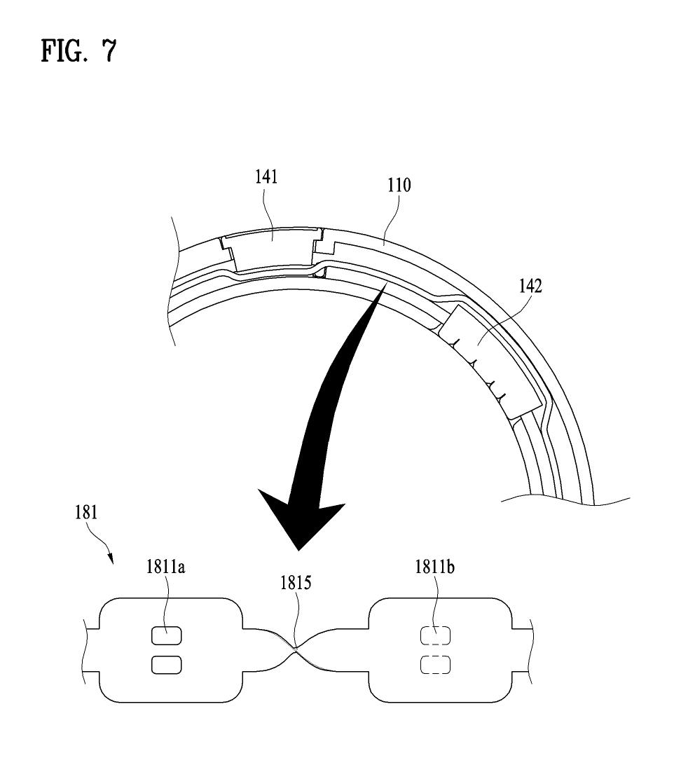

FIG. 7 is a diagram illustrating a flexible board 181 of a portable electronic device 100 according to the present invention. The flexible board 181 corresponds to a member where a circuit pattern including an access terminal 1811 is formed on a surface of an insulation board. The access terminal 1811 is connected with various components and a signal can be transmitted and received between components according to the circuit pattern.

The flexible board 1811 is deployed in the inner side of the first housing 110 of a ring shape and an access surface of each component and the access terminal 1811 exposed to a surface of the flexible board 181 are electrically connected. In general, a front side of each component is exposed to the outer side of the first housing 110 and an access face accessed with the flexible board 181 is deployed in the rear side of each component. In order to minimize thickness of the portable electronic device 100, a rear side (access face) of a component positioned at the outer side of the first housing 110 and a rear side (access face) of a component positioned at the inner side of the first housing 110 are positioned at a different height in thickness direction of the first housing 110.

In order to connect each of the components of which a height of an access face is different from each other, the flexible board 181 is deployed in a manner of being bent. In this case, since an access face of a component positioned at the outer side of the first housing 110 faces the inner side of the first housing 110 and an access face of a component positioned at the inner side of the first housing 110 faces the outer side of the second housing 120, a face where each component is contacted with the flexible board 181 may have an opposite direction.

In particular, the flexible board 181 forms a different pattern on both sides. A component positioned at the outer side of the first housing 110 accesses a first side of the flexible board 181 and a component positioned at the inner side of the second housing 120 may access a second side of the flexible board 181. However, if a different pattern is formed on both sides of the flexible board 181, it may have a problem that a manufacturing cost increases.

In order to solve the problem, an access terminal 1811 contacted with each component is located at one side of the flexible board 181 and, as shown in FIG. 7, the flexible board 181 can be deployed in a manner of being twisted. Since a twisted part 1815 of the flexible board 181 corresponds to a part in which a component is not deployed, there is no problem in deploying the twisted part 1815.

If a width of the flexible board 181 of the twisted part 1815 is narrowly formed compared to a width of a part at which each of access terminals is positioned, it may be able to minimize the increase of thickness of the twisted part 1815. In some cases, the twisted part 1815 can be deployed in a state of being pushed and folded.

As mentioned in the foregoing description, since it is able to minimize a battery size of a portable electronic device 100 and simply perform self-charging, it is able to use the portable electronic device for a long time.

And, since a sensor located at the inside of a portable electronic device is combined with a body of a user in a manner of being adhere to the body, it is able to consistently measure bio-information such as an electrocardiogram, a body temperature, and the like.

In the foregoing description, embodiments related to a control method capable of being implemented in the portable electronic device 100 have been explained with reference to the attached drawings. It will be apparent to those skilled in the art that various modifications and variations can be made therein without departing from the spirit and scope of the invention.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds, are therefore intended to be embraced by the appended claims.

What is claimed is:

1. A portable electronic device, comprising:
a first housing of a ring shape;
a second housing having a ring shape corresponding to the first housing and is arranged to a side of the first housing in parallel;
a charging coil embedded in the first housing and wound along the ring shape of the first housing;
a plurality of magnets deployed to the second housing; and
if the second housing rotates with respect to the first housing, a battery charged using a current flowing through the charging coil.

2. The portable electronic device of claim 1, further comprising ball bearings which are positioned between the first housing and the second housing to assist the second housing to rotate.

3. The portable electronic device of claim 1, wherein an inner side of the second housing comprises a material of low friction force and wherein an inner side of the first housing comprises a material of high friction force.

4. The portable electronic device of claim 1, wherein a size of a circle formed by an inner side of the second housing is greater than a size of a circle formed by an inner side of the first housing.

5. The portable electronic device of claim 2, wherein an inner side of the second housing has a slope inclined to an outer side of the second housing as getting far from the first housing.

6. The portable electronic device of claim 1, wherein the second housing is deployed to both sides of the first housing as a pair.

7. The portable electronic device of claim 1, further comprising:
a first part mounted in a manner of being adjacent to an outer side of the first housing or the first part mounted in a manner of being exposed to the outer side;
a second part mounted in a manner of being adjacent to an inner side of the first housing or the second part mounted in a manner of being exposed to the inner side; and
a flexible board configured to connect the first part, the second part, and the battery while being positioned at the inside of the first housing,
wherein a part of the flexible board overlapped with the first part is bent in a direction of the inner side of the first housing and wherein a part of the flexible board overlapped with the second part is bent in a direction of the outer side of the first housing.

8. The portable electronic device of claim 7, wherein the flexible board comprises an insulation film, a first electrode formed on one side of the insulation film, and a second electrode formed on one side of the insulation film in a manner of being separated in a length direction of the insulation film and wherein the first electrode and the second electrode are respectively contacted with the first part and the second part in a manner that the insulation film is twisted between the first electrode and the second electrode to make the first electrode and the second electrode face an opposite direction.

9. The portable electronic device of claim 1, further comprising a plurality of body fat measuring terminals formed on an outer side of the first housing.

10. The portable electronic device of claim 1, further comprising an electrocardiogram sensor formed on an inner side of the first housing.

11. The portable electronic device of claim 9, wherein the electrocardiogram sensor comprises a pair of electrocardiogram sensor terminals deployed to the inner side of the first housing to make the terminals face each other.

12. The portable electronic device of claim 1, further comprising:
a wireless communication unit configured to connect an external terminal;
a plurality of LEDs mounted on an outer side of the first housing; and
if event information is received via the wireless communication unit, a controller configured to control the event information to be provided to a user by controlling color, a turn-on order, a turn-on count, and the like of a plurality of the LEDs.

13. The portable electronic device of claim 1, further comprising a controller configured to supply a current flowing through the charging coil to the battery in a charging mode and the controller configured to control the portable electronic device according to a direction or an amount of the current flowing through the charging coil in a user input mode.

14. The portable electronic device of claim 13, further comprising a wireless communication unit configured to connect an external terminal, wherein if event information is received via the wireless communication unit, the controller is configured to switch a mode into the user input mode.
